(19) United States
(12) Patent Application Publication
(10) Pub. No.: US 2021/0071264 A1
(43) Pub. Date: Mar. 11, 2021

(54) EXPRESSION AND GENETIC PROFILING FOR TREATMENT AND CLASSIFICATION OF DLBCL

(71) Applicant: Genomic Testing Cooperative, LCA, Irvine, CA (US)

(72) Inventor: Maher Albitar, Valley Center, CA (US)

(73) Assignee: Genomic Testing Cooperative, LCA, Irvine, CA (US)

(21) Appl. No.: 17/018,347

(22) Filed: Sep. 11, 2020

Related U.S. Application Data
(60) Provisional application No. 62/899,007, filed on Sep. 11, 2019.

(51) Int. Cl. C12Q 1/6886; C12Q 1/6827; G01N 33/50

(52) U.S. Cl. CPC C12Q 1/6886; C12Q 1/6827; C12Q 2600/156; C12Q 2600/158; G01N 33/5082

(57) ABSTRACT

A method of treating diffuse large B-cell lymphoma, comprises obtaining a sample from a patient having diffuse large B-cell lymphoma; detecting in the sample, by an assay, mutation in each gene in a first panel; quantifying in the sample an expression level of each gene in a second panel; classifying the diffuse large B-cell lymphoma of the patient as having a cell of origin of either (i) germinal-center B-cell-like or (ii) activated B-cell-like; and treating the patient with a cancer treatment therapy regime. The first panel comprises at least one gene selected from the group consisting of EZH1 and MYD88; and the second panel comprises at least one gene selected from the group consisting of IRF4, MYBL1, RASGRF1, S1PR2 and SSBP2.

### CROSS REFERENCE TO RELATED APPLICATION

This application claims the benefit of U.S. Provisional Application No. 62/899,007, filed 11 Sep. 2019, entitled “CELL OF ORIGIN CLASSIFICATION OF DLBCL USING TARGETED NGS EXPRESSION PROFILING AND DEEP LEARNING”, attorney docket no. 104183.0001PRO, the contents of which are hereby incorporated by reference in their entirety, except where inconsistent with the present application.

### BACKGROUND

Diffuse large B-cell lymphoma (DLBCL) is the most common B-cell lymphoma and is clinically heterogeneous. Gene expression profiling (GEP) classified DLBCL into 2 major molecular subtypes according to their cell of origin (COO): germinal-center B-cell-like (GCB) and activated B-cell-like (ABC) DLBCL.¹ ABC-COO is associated with poorer clinical outcomes in DLBCL irrespective of treatment: CHOP (cyclophosphamide, doxorubicin, vincristine, and prednisone), rituximab (R)-CHOP,¹⁻³ obinutuzumab (G)-CHOP,⁴ or classical salvage chemotherapy R-DHAP (rituximab, dexamethasone, high-dose cytarabine, and cisplatin) followed by intensive therapy plus autologous stem cell transplantation.⁵ However, several novel agents, including lenalidomide,⁶⁻⁸ ibrutinib,^{8,9} and bortezomib alone¹⁰ or in combination with durvalumab (anti-PD-L1),¹¹ showed selective or better clinical efficacy in ABC- vs GCB-DLBCL. The prognostic and therapeutic differences between ABC- and GCB-DLBCL have a molecular basis, such as higher frequencies of mutations in CD79, MYD88, CARD11, PRDM1, and TNFAIP3,¹² chronic active B-cell receptor signaling,¹³ and more frequent MYC/BCL2 double expression in the absence of genetic MYC/BCL2 double hit¹⁴ in ABC-DLBCL. In addition, the subcellular distribution and mechanism of action of doxorubicin in ABC-DLBCL are different from those in GCB-DLBCL.¹⁵ To guide clinical therapeutics, distinction of the GCB vs ABC/non-GC subtype has become the standard practice according to the 2016 revision of the World Health Organization classification of lymphoid neoplasms.¹⁶

Significant efforts have been put into establishing clinically applicable assays and accurate classification of DLBCL, and methodology to determine COO has been evolving in the last 2 decades. The original Lymphochip spotted cDNA microarray and the gold standard classification algorithm are robust in COO classification but impracticable for routine clinical practice.¹⁻³ Researchers thus developed algorithms to distinguish GC from non-GC subtypes based on protein expression of 3 to 5 biomarkers in formalin-fixed, paraffin-embedded (FFPE) tissue samples readily assessed by immunohistochemistry (IHC) in the clinic.¹⁷⁻²⁴ However, the accuracy of these IHC algorithms and the prognostic significance of COO subtypes determined by IHC algorithms^{5,25} are not consistent.^{23,26-28} To enable GEP by DNA microarrays to classify DLBCL using clinical FFPE tissues that yield highly fragmented RNA samples, new RNA amplification and labeling techniques and classification models were developed, including a 100-gene classifier for Affymetrix GeneChip (Affymetrix, Inc) data²⁹ and a 20-gene DLBCL Automatic classifier for Illumina WG-DASL platform (Illumina United Kingdom) data³⁰ developed from a previous platform-independent 27-gene DLBCL subgroup predictor³¹ that showed reproducibility and prognostic value.

To simplify the GEP process for FFPE samples, a multiplexed quantitative nuclease protection assay (qNPA) was developed that directly hybridizes mRNA in situ using 50-mer probes for genes of interest, followed by probe capture and quantitative imaging, thereby reliably detecting mRNA levels in FFPE samples without RNA exaction and amplification.³²⁻³⁴ The qNPA platform (HTG Molecular Diagnostics, Inc.) can accurately classify DLBCL using a 14-gene signature.³⁵ The current HTG EdgeSeq DLBCL COO assay has been applied in a clinic trial.³⁶ However, the most successful simplified variation of microarray for rapid COO determination is the NanoString nCounter System (NanoString Technologies), which elegantly detects target mRNA of interest in extracted nonamplified RNA samples using a capture probe and a color-coded reporter probe, followed by purification, immobilization, and digital readout.³⁷ Several different small gene panel-based DLBCL-COO assays, including the most wildly used Lymph2Cx 20-gene assay,³⁸ have been applied in research studies and clinical trials,^{4,39-45} although a large gene panel (145 genes) was also achievable for the NanoString nCounter system.⁴⁶ COO determined by Lymph2Cx 20-gene assay either exhibited high concordance with GEP-determined COO or showed significant prognostic value in 4 retrospective studies⁴⁷⁻⁵⁰ and a clinical tria1,⁵¹ but not in 2 clinical trials⁵² and 1 retrospective study.⁵³

Reverse transcriptase-multiplex ligation-dependent probe amplification, which ligates the left and right probes annealed to cDNA target sequences, permitting amplification of specific genes,⁵⁴ is another type of assay that has been applied for DLBCL-COO classification based on expression of 14 or 21 genes.^{55,56} This method is sensitive and cost-effective without using a dedicated platform but has relatively poor dynamic range and is unable to include some COO-specific genes.⁵⁵

DLBCL outcome predictors that link GEP signatures directly to clinical outcome instead of COO have also been developed,^{2,3,57,58} but the reproducibility between different studies was poor, and the predictive value for therapies other than the standard treatment is uncertain. In contrast, COO classification with underlying biology basis⁹ also have predictive values for novel therapies, as demonstrated in phase 1/2 and 2/3 clinical trials.^{6-8,10,11} However, recent clinical trials for adding ibrutinib (phase 3³⁶) and bortezomib (phases 2⁵⁹ and 3⁶⁰) to the standard R-CHOP in previously untreated ABC (by Hans algorithm and HTG EdgeSeq³⁶ or by Illumina DASL assay⁶⁰) or non-GC (by Hans algorithm and Nanostring Lymph2Cx assay⁵⁹) DLBCL patients failed to show improved clinical outcome.

To better classify DLBCL biologically guiding therapeutic clinical trials, genetic alteration signatures have been explored to subtype DLBCL in large numbers of patients, as genetic upstream of the oncogenic biology in DLBCL can define the response to novel targeted therapies. Schmitz et al⁶¹ used a GenClass algorithm, and Chapuy et al⁶² used an nonnegative matrix factorization (NMF) consensus clustering algorithm to analyze high-content genetic data of 574 and 304 patients, respectively, and uncovered genetically distinct subtypes within or independent of COO subtypes, most of which demonstrated robust prognostic significance and potential therapeutic relevance.^{61,62} However, the pathogenic driver roles of many mutations in signatures vary or have not been validated,^{63,64} and how to accurately assign a genetic subtype to new individual patients at presentation in real time is less clear than the current COO classification. In a phase 3 GOYA study (NCT01287741),⁴³ approximation of EZB, BN2, N1, and MCD subtypes based on presence of subtype founder gene alterations in targeted next-generation sequencing (NGS) data of 465 genes did not find prognostic effect, whereas clusters (C) C2, C3, and C5 identified by applying NMF consensus clustering to the study cohort showed poorer prognosis compared with C0, C1, and C4 clusters. In another prospective study from the LNH03B LYSA (Lymphoma Study Association) clinical trials with targeted NGS of 34 key genes and genomic copy number variation analysis, none of the genetic subtypes identified by the GenClass algorithm or NMF consensus clustering showed prognostic significance.⁶⁵ The inconsistent prognostic values could result from the highly variable sequencing panels and NGS data quality in different studies, inaccurate subtyping, and the clinical heterogeneity within defined genetic subtypes underscored by phenotypic biologic (eg, MYC/BCL2 expression⁶⁶) heterogeneity arising from many other underlying mechanisms, for example, epigenetic deregulation and genetic alterations in noncoding regions.⁶⁷ In fact, in the cohort of Schmitz et al, MCD patients with MYD88/CD79B double mutations had better survival compared with other MCD patients,⁶⁶ and the EZB subtype has been further divided into the unfavorable EZB-MYC+ and favorable EZB-MYC⁻ subtypes recently by a LymphGen algorithm.⁶⁸ A LymphGen webtool has been public accessible and able to assign genetic subtypes to patients if the input is from a cohort but not if from only 1 patient.

### SUMMARY

In a first aspect, the present invention is a method of treating diffuse large B-cell lymphoma, comprising obtaining a sample from a patient having diffuse large B-cell lymphoma; detecting in the sample, by an assay, mutation in each gene in a first panel; quantifying in the sample an expression level of each gene in a second panel; classifying the diffuse large B-cell lymphoma of the patient as having a cell of origin of either (i) germinal-center B-cell-like or (ii) activated B-cell-like; and treating the patient with a cancer treatment therapy regime. The first panel comprises at least one gene selected from the group consisting of EZH1 and MYD88; and the second panel comprises at least one gene selected from the group consisting of IRF4, MYBL1, RASGRF1, S1PR2 and SSBP2.

In a second aspect, the present invention is a method, comprising detecting in a sample from a patient having diffuse large B-cell lymphoma, by an assay, mutation in each gene in a first panel; and quantifying in the sample an expression level of each gene in a second panel. The first panel comprises at least one gene selected from the group consisting of EZH1 and MYD88; and the second panel comprises at least one gene selected from the group consisting of IRF4, MYBL1, RASGRF1, S1PR2 and SSBP2.

In a third aspect, the present invention is a method, comprising detecting in a sample from a patient having diffuse large B-cell lymphoma, by an assay, mutation in each gene in a first panel; and quantifying in the sample an expression level of each gene in a second panel. The first panel comprises TP53; and the second panel comprises at least one gene selected from the group consisting of CARD11, BCL6, MALAT1, RABEP1 and BCORL1.

In a fourth aspect, the present invention is a method, comprising detecting in a sample from a patient having diffuse large B-cell lymphoma, by an assay, mutation in each gene in a first panel; and quantifying in the sample an expression level of each gene in a second panel. The first panel comprises TP53; and the second panel comprises at least one gene selected from the group consisting of CDK8, LMO2, BCR, TGFBR2, CHD2 and ETS1.

### BRIEF DESCRIPTION OF THE DRAWINGS

FIGS. 1A, 1B and 1C Two-dimensional representation of the training and validation set data using the autoencoder. (A) Training set data using the autoencoder. (B) Validation set data using the autoencoder. (C) NanoString validation data using the autoencoder.

FIGS. 2A and 2B: GCB and ABC subtypes defined by the new NGS-COO classifier.

FIGS. 3A, 3B, 3C and 3D: Risk stratification of DLBCL patients by the new NGS-survival risk scores from Cox proportional hazards models based on two latent features of the data obtained from the autoencoders. (A, B) OS curves of 3 risk groups defined by the NGS-OS risk scores. (C, D) PFS curves of 3 risk groups defined by the NGS-PFS risk scores.

FIG. 4: Schematic representation of the deep learning approach.

### DETAILED DESCRIPTION

Based on these previous studies, we hypothesized that combined high-throughput genetic and gene expression signature analysis may improve the DLBCL classification for prognostic stratification and therapeutic implication. To be clinically applicable, fast and economical assays on FFPE samples that provide both genetic and expression data with low sample input are needed. We therefore implemented targeted RNA sequencing (RNA-Seq) of 1408 genes with NGS technology that simultaneously sequences and quantitates expressed mRNA molecules in a single assay. Artificial intelligence (AI) was implemented to build predictive models based on both genetic and gene expression data of a large number of DLBCL FFPE samples. The robustness of the predictive models was tested in validation cohorts supporting our hypothesis. The full details of the study have been published⁸⁴ (Xu-Monette Z Y, et al. A refined cell-of-origin classifier with targeted NGS and artificial intelligence shows robust predictive value in DLBCL. Blood Advances 2020; 14(4):3391), the contents of which are hereby incorporated by reference in their entirety, except where inconsistent with the present application. Three models were developed: a model for classifying the cell of origin (COO) of the DLBCL, a prognostic model for DLBCL overall survival (OS), and a prognostic model for DLBCL progression-free survival (PFS).

The model for classifying the COO of the DLBCL includes detecting in a sample from a patient mutation in each gene in a first COO panel, and quantifying expression levels of each gene in a second COO panel. Each COO panel includes at least one gene. The first COO panel may include one or more of the genes EZH1 and MYD88. Preferably the first COO panel includes EZH1, and more preferably both EZH1 and MYD88. The second COO panel may include one or more of the genes AFF3, AHR, AUTS2, BCAS4, BCL6, BTLA, CARD11, CCND2, CCND3, CD22, CD44, COL9A3, CREB3L2, EBF1, ETV6, FAM46C, FOXP1, IKZF1, IL2RA, IRF4, IRS1, KANK1, LCK, LMO2, LPP, LRMP, LRP5, LRRK2, LYL1, LYN, METTL7B, MYBL1, P2RY8, PAG1, PAK6, PDGFD, PIK3CG, PIM1, PTK2, PTK2B, PTPN2, RASGRF1, S1PR2, SSBP2, STAT3 and TBL1XR1. Preferably, the second COO panel includes one or more of IRF4, MYBL1, RASGRF1, 51PR2 and SSBP2, and more preferably the second COO panel includes all 5 of these genes. The second COO panel may include 1 to 46 genes including 2, 3, 4, 5, 10, 15, 20, 25, 30, 35, 40 or 45 genes.

Optionally, the COO of the DLBCL may be identified, and then optionally the clinician can select the most appropriate cancer treatment therapy regime. Preferably, the confidence of classifying the COO is a probability of at least 0.8. Examples of cancer treatment therapy regimes include administering one or more of cyclophosphamide, doxorubicin, vincristine, prednisone, rituximab, obinutuzumab, dexamethasone, cytarabine, cisplatin, lenalidomide, ibrutinib, bortezomib, durvalumab and autologous stem cell transplantation. The best selection of treatment of DLBCL based on the COO of the DLBCL may be found, for example in UpToDate, a clinical decision support resource that is used to aid medical professionals in diagnosing and making treatment decisions (UpToDate, Wolters Kluwer, www.uptodate.com/home).

The prognostic model for DLBCL OS includes detecting in a sample from a patient mutation in each gene in a first OS panel, and quantifying expression levels of each gene in a second OS panel. Each OS panel includes at least one gene. The first OS panel may include one or more of the genes TP53 and TET2. Preferably the first OS panel includes TP53, and more preferably both TP53 and TET2. The second OS panel may include one or more of the genes AFF3, ASPSCR1, BCL2, BCL6, BCORL1, BHLHE22, BTK, CARD11, CCND2, CD58, CHEK2, CIT, CREB3L2, DST, ETS1, EYA2, FANCF, FZD6, GAS5, HMGA1, HOXA9, IRF4, KDM5C, KLK2, LFNG, LMO2, MACROD1, MALAT1, MEF2B/MEF2BNB-MEF2B, MFNG, MLLT4, MTCP1, MYC, PIM1, POLD1, PPP3CA, RABEP1, RAD51B, RBM6, RECQL4, RHBDF2, RLTPR, RTEL1-TNFRSF6B, SMAD3, SPTBN1, SRRM3, ST6GAL1, SULF1, SYP, TEAD2, TFAP2A, TGFBR3, U2AF2 and ZIC2. Preferably, the second OS panel includes one or more of CARD11, BCL6, MALAT1, RABEP1 and BCORL1, and more preferably the second OS panel includes all 5 of these genes. The second OS panel may include 1 to 54 genes including 2, 3, 4, 5, 10, 15, 20, 25, 30, 35, 40, 45, 50 or 53 genes. Preferably, the age of the patient (that is, the patient being over or under 60 years of age) is also included as a factor in determining OS.

The prognostic model for DLBCL PFS includes detecting in a sample from a patient mutation in each gene in a first PFS panel, and quantifying expression levels of each gene in a second PFS panel. Each PFS panel includes at least one gene. The first PFS panel includes TP53. The second PFS panel may include one or more of the genes AFF1, AFF3, ASPSCR1, ATM, BCL2, BCR, BTG2, BTK, BTLA, CDK12, CDK8, CHD2, CHEK2, CIRH1A, CREB3L2, DDIT3, EDNRB, EPHB6, ETS1, FANCF, FOXP1, FZD6, GAB1, GAS5, GPR34, IQCG, ITGA7, KDM5C, KDSR, LAMA5, LFNG, LIFR, LMO2, MACROD1, MAP2K5, MFNG, MYC, NCSTN, NR6A1, POU2AF1, PRKCB, RLTPR, RPL22, SHC2, SMAD3, SPTBN1, ST6GAL1, TEAD2 and TGFBR2. Preferably, the second PFS panel includes one or more of CDK8, LMO2, BCR, TGFBR2, CHD2 and ETS1, and more preferably the second PFS panel includes all 6 of these genes. The second PFS panel may include 1 to 49 genes including 2, 3, 4, 5, 10, 15, 20, 25, 30, 35, 40, 45 or 48 genes.

A sample from a patient may be a tissue sample (such as a tumor tissue sample or bone marrow tissue sample) or a cell free RNA sample. The sample may be fresh tissue or formaldehyde-fixed paraffin-embedded (FFPE) tissue. Preferably, the patient has already been diagnosed with DLBCL. A variety of techniques to detect mutation in genes and the expression level of genes in a sample are known. Preferably, both detection of the mutations and the expression levels are determined using next generation sequencing (NGS) in a single assay. Preferably, all the genes of interest in all 3 models, both mutations and expression levels, are determined in a single assay. Preferably, the expression levels are normalized, for example normalized to the expression level of the PAX5 gene.

### Examples

### Patients and Methods

### Patients

RNA-seq was performed for 444 patients with de novo DLBCL diagnosed in 1998 to 2008 treated with R-CHOP at 22 medical centers. Cases were organized for retrospective studies as part of the DLBCL Consortium Program,⁶⁹ which has been approved by the institutional review board of each participating medical center and conducted in accordance with the Declaration of Helsinki. Patients with transformed DLBCL, primary mediastinal large B-cell lymphoma, primary central nervous system DLBCL, or primary cutaneous DLBCL have been excluded. Molecular characterization of the study cohort has been previously summarized.^{70,71} Fluorescence in situ hybridization identified 12 of 293 cases as high-grade B-cell lymphoma with MYC and BCL2 and/or BCL6 rearrangements (7 MYC/BCL2 double/triple-hit and 5 MYC/BCL6 double-hit cases).

Data for 418 cases were further analyzed after data quality control. GEP was performed in 366 of the 418 patients using Affymetrix GeneChip Human Genome U133 Plus 2.0 (deposited in Gene Expression Omnibus GSE #31312).²⁴ Using a Bayesian model, 172, 160, and 34 cases were determined as GCB, ABC, and unclassified DLBCL, respectively. For the 34 GEP-unclassified cases, the Visco-Young IHC algorithm²⁴ was applied, which assigned 15 cases to GCB and 19 cases to ABC. For the other 52 cases in which GEP was not performed, the Visco-Young algorithm classified 22 cases as GCB and 23 cases as ABC.

To further validate the COO model, 60 independent DLBCL samples were obtained and classified into ABC/GCB subtypes using the Lymph2Cx NanoString nCounter assay according to the manufacturer's instructions.

### GEP Analysis

Raw RNA-Seq and Affymetrix GEP data were preprocessed and normalized by robust multichip average using the R package (version 1.65.1).⁷² Two-class unpaired significance analysis of microarrays were performed to identify significantly differentially expressed genes (DEGs) between the 2 groups.⁷³ Gene expression data were analyzed via CLUSTER software using the average linkage metric and then displayed by JAVA TREEVIEW (www.java.com/en).⁷⁴

### RNA Library Construction and Sequencing

The Agencourt FormaPure Total 96-Prep Kit was used to extract both DNA and RNA from the same FFPE tissue lysates using an automated KingFisher Flex and protocols as recommended by each manufacturer. Samples were selectively enriched for 1408 cancer-associated genes using reagents provided in an IIlumina TruSight RNA Pan-Cancer Panel. The cDNA was generated from the cleaved RNA fragments using random primers during the first- and second-strand synthesis. Then, sequencing adapters were ligated to the resulting double-stranded cDNA fragments. The coding regions of expressed genes were captured from this library using sequence-specific probes to create the final library. Sequencing was performed on an IIlumina NextSeq 550 System platform. Ten million reads per sample in a single run was required. The read length was 2×150 bp. The sequencing depth was 10× to 1739×, with a median of 41×. An expression profile was generated from the sequencing coverage profile of each individual sample using Cufflinks. Expression levels were measured using fragments per kilobase of transcript per million and further normalized using the B-cell PAX5 RNA expression levels to adjust for variability in the percentage of DLBC cells in samples.

Alignment of sequencing data and variant calling were performed with the DRAGEN Somatic Pipeline (IIlumina) using tumor-only analysis against the GRCh37 reference genome to identify 2 classes of mutations: single nucleotide variants and indels. Tumor samples were analyzed without a matching normal.

### DLBCL COO Classification and Clinical Risk Prediction Modeling

To build robust DLBCL classification models, we randomly selected 60% of cases to fit (train) the model and then validated using the remaining 40% (validation set). Sixty independent DLBCL samples classified by Nanostring Lymph2Cx assay were used as a second validation set.

First, univariate significance tests were used to screen the large number of variables. Normalized RNA expression data and mutation data were included as variants to build a classification model. For interpretability and simplicity, we divided the gene expression values into 4 or 10 equal parts using the quartiles (Q1, Q2, and Q3) and deciles and selected mutation data of 39 highly recurrent genes that had mutations in at least 10 patients. Fisher's exact test was used after discretizing RNA expressions using their quartiles, and 228 variables were statistically significant with P<0.01. After adjusting for multiple hypothesis testing using Benjamini-Hochberg's method and setting the cutoff for false discovery rate (FDR) at 0.01, statistically significant variables were narrowed down to 129. Finally, setting the cutoff for FDR at 0.0001, 48 variables were selected with either small adjusted P values or high area under the receiver operating curve (AUC).

We selected 252 DLBCLs with high confidence COO assignment to develop risk stratification models directly correlating with survival. We randomly selected 60% (152) of subjects as the training set to fit the model and tested the performance in the remaining 40% (100) patients. Kaplan-Meier and Cox proportional hazards (CPH) analysis was used to identify variables with significant prognostic impact.

Multiple statistical approaches were tested for modeling performance, and models built through deep learning techniques^{75,76} were most predictive and robust. We used autoencoders for nonlinear transformations of autoencoded features into 2-dimensional latent space. Logistic regression and CPH models were used for building the COO model and clinical risk models, respectively.

### Results

### Prognostic Significance in DLBCL

Mutation status of each gene was analyzed for prognostic significance. Table 2 lists frequently mutated genes with significant mutational effects on overall survival (OS) by univariate analysis. The DLBCL group had 418 patients. The GCB group, determined by gene-expression profiling, had 172 patients. The ABC group, determined by gene-expression profiling, had 160 patients. The impact on OS was based on univariate analysis for each gene. Among genes with mutations occurring in at least 9 patients, TP53, TET2, KMT2D (in overall cohort, P=0.0005, 0.011, and 0.012, respectively), NOTCH2 (in GCB, P=0.005), and ATM (in ABC, P=0.003) mutations showed significantly adverse effects, whereas EZH2 and GNA13 mutations genes showed significantly favorable effects (P=0.007 and 0.047, respectively).

TABLE 2
List of genes with >2% mutational prevalence
and significant impact on OS rate in DLBCL

| | | In DLBCL | | In GCB | | In ABC | |
|---|---|---|---|---|---|---|---|
| | | Mutation | P for | Mutation | P for | Mutation | P for |
| Gene | Effect on OS | frequency, % | OS | frequency, % | OS | frequency, % | OS |
| KMT2D | Unfavorable | 23.7 | .012 | 28.5 | .005 | 17.5 | — |
| TP53 | Unfavorable | 16.3 | .0005 | 19.8 | .024 | 13.1 | .034 |
| EZH2 | Favorable | 9.3 | .007 | 17.4 | .04 | 1.9 | — |
| TET2 | Unfavorable | 5.5 | .011 | 6.4 | .011 | 4.4 | — |
| NOTCH2 | Unfavorable | 4.5 | (.061) | 2.9 | .005 | 6.9 | — |
| GNA13 | Favorable | 4.1 | .047 | 8.1 | (.09) | 0.6 | — |
| ATM | Unfavorable | 2.2 | (.085) | 1.7 | — | 2.5 | .003 |

Marginal P values are in the parentheses. Dashes indicate not prognostic.

### Development and Validation of the NGS-COO Classification Model

RNA-Seq gene expression⁸⁴, gene fusion, and mutation data were used to develop a model for DLBCL-COO classification in the training set. Fisher's exact test and multiple hypothesis testing adjustment were used to identify RNA-Seq variables showing significant difference between GCB and ABC subtypes. Finally, the top 48 variables (Table 3) that were significantly differed between GCB and ABC subtypes with FDR<0.0001 or high AUC were chosen to build a new classification model for RNA-Seq data, including 2 genes (MYD88 and EZH2)'s mutation status and 46 genes' RNA expression levels.

TABLE 3
List of 48 variables (46 for gene expression level and 2
for gene mutation status) in the DLBCL NGS-COO classifier

| | | | | | |
|---|---|---|---|---|---|
| AFF3 | AHR | AUTS2 | BCAS4 | BCL6 | BTLA |
| CARD11 | CCND2 | CCND3 | CD22 | CD44 | COL9A3 |
| CREB3L2 | EBF1 | ETV6 | FAM46C | FOXP1 | IKZF1 |
| IL2RA | IRF4 | IRS1 | KANK1 | LCK | LMO2 |
| LPP | LRMP | LRP5 | LRRK2 | LYL1 | LYN |
| METTL7B | EZH1 mutation | MYD88 mutation | MYBL1 | P2RY8 | PAG1 |
| PAK6 | PDGFD | PIK3CG | PIM1 | PTK2 | PTK2B |
| PTPN2 | RASGRF1 | S1PR2 | SSBP2 | STAT3 | TBL1XR1 |

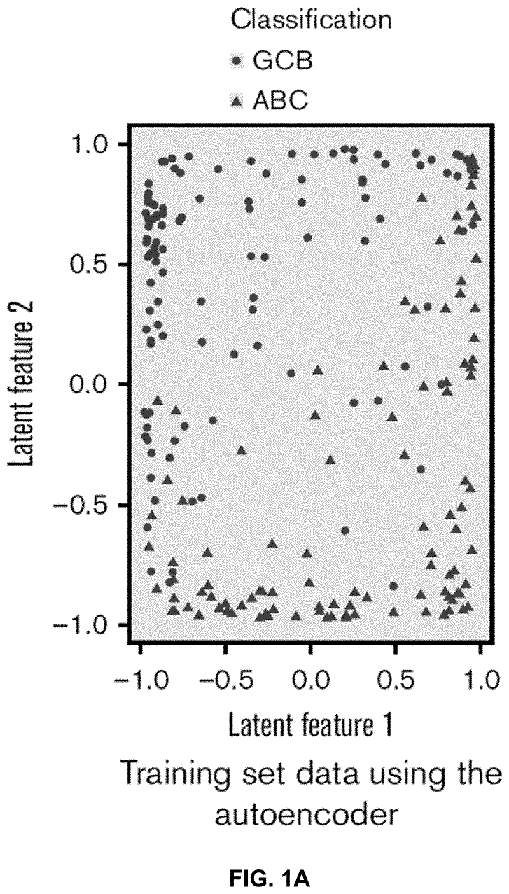

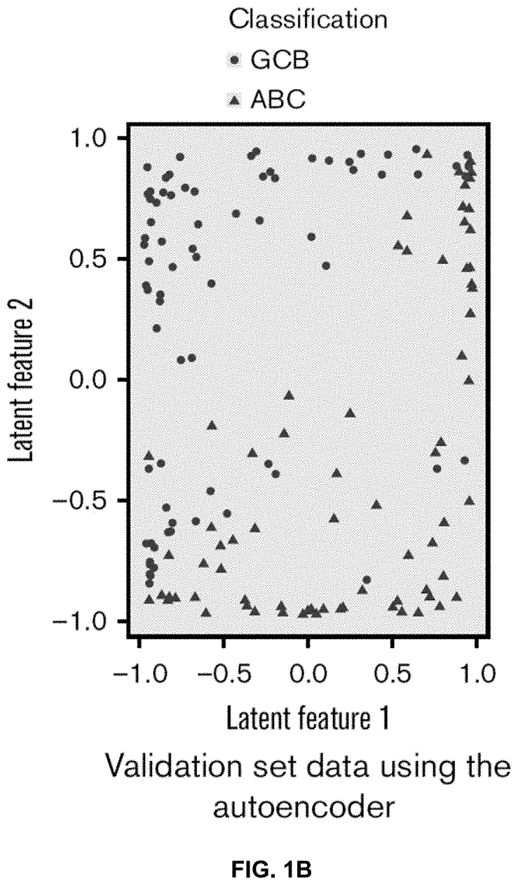

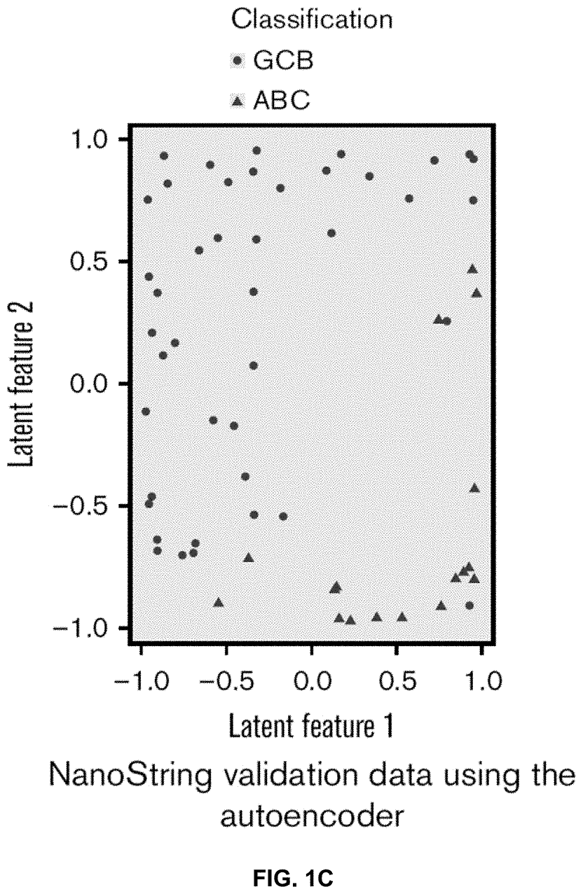

Several statistical models were built on the 48 variables in the training set (without knowing classification) and then tested in the validation sets. The COO model based on autoencoder, an unsupervised deep learning technique, showed the best performance. An autoencoder neural network was built with 5 hidden layers.^{75,76} The first 2 layers and the last 2 layers each had 100 neurons; the middle layer (bottleneck) had 2 neurons, which captured latent (unobserved) features of the data. The values of these 2 neurons formed a low-dimensional (2) representation of the data; that is, it aggregated the 48 variables into 2 latent features. The top 7 contributing variables to the latent features were MYD88 mutation, EZH2 mutation, RASGRF1 expression, MYBL1 expression, S1PR2 expression, SSBP2 expression, and IRF4 expression. Based on the latent features, a logistic regression model was built for GCB/ABC classification (named as NGS-COO classifier). As shown in FIG. 1A, the autoencoder transformed the high-dimensional data into a 2-dimensional space where the 2 subtypes were easily separable (linearly) roughly with a diagonal line from (−1, −1) to (1, 1).

The NGS-COO classifier developed from the training set was then applied to the validation set. A probability of scoring was generated for each case. Approximately 30% of the cases had a score between 0.5 and 0.75, indicating low confidence for classification. For the remaining 70% with high confidence for assigning to 1 of the 2 subtypes (probability of 0.8 or higher), the ABC vs GCB classification showed sensitivity and specificity of 96% and 97% for classification in the validation set. The accuracy/concordance rate with previous GCB/ABC classification was 95.6%. The corresponding AUC was 96.2%.

In the training and validation sets, in total, 216 cases were determined as the ABC subtype and 202 cases as the GCB subtype. The new GCB/ABC cases were also associated with 1319 significant DEGs with FDR<0.0001 in GEP analysis using our previous Affymetrix GeneChip DNA microarray data and multiple biomarkers characterized in previous studies by our Consortium program.

To further evaluate the performance of the NGS-COO classification model, we applied the same approach to 60 independent cases as an external validation cohort. Our NGS-COO model showed sensitivity and specificity of 96% and 97%, respectively, with the previous COO classification by the NanoString Lymph2Cx assay. The concordance rate was 92.9%. The corresponding AUC was 95.7%. As shown in FIG. 1B-C, the pattern of separation by the autoencoder was similar between training, validation, and independent sets. They all showed separation between ABC and GCB with a diagonal line from (−1, −1) to (1,1), although the independent 60 cases were collected from a completely different set of samples and the sequencing was performed separately.

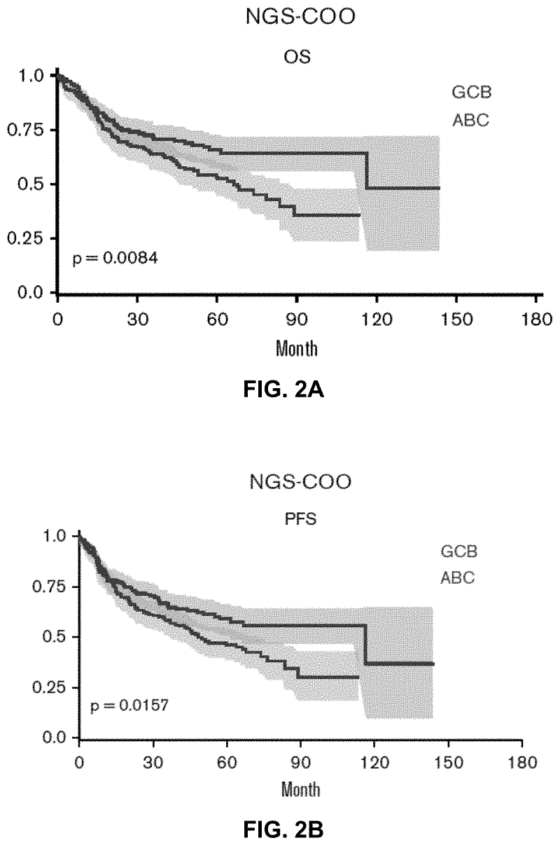

The performance of our NGS-COO classifier was also evaluated by correlating with survival outcomes. Although the autoencoder was only trained for COO classification in the training set, the NGS-COO classifier was significantly associated with OS and progression-free survival (PFS) in DLBCL, similar to the previous COO classification (FIG. 2A-B). The relative risk of the new ABC compared with the new GCB group was 1.53. The prognostic significance was slightly improved if comparing within high-confidence cases only (risk for OS, 1.81, P=0.007; risk for PFS, 1.77, P=0.0046).

### Development of Prognostic Models for DLBCL Risk Stratification

To build robust prognostic models aggregating small contributions of a large number of variables directly to patient survival, we used a similar procedure and the AI method to develop models in the training set and test the performance in the validation set based on both gene expression and genetic variables plus 2 additional factors: age and sex of patients. We first screened for significant variables using Kaplan-Meier and CPH for OS in the training set. Although 61 variables showed significant prognostic effects by log-rank test and 110 variables by CPH regression (P<0.05), only the TP53 mutation remained statistically significant after adjusting for multiple hypothesis testing (FDR<0.0001). Therefore, we selected 57 variables with the top 2% AUC values or P<0.01 (either based on log-rank test or CPH; Table 4).

TABLE 4
List of 57 variables (54 for gene expression level, 1 for age over 60 and
2 for gene mutation status) selected for building the NGS-OS risk model

| | | | | | |
|---|---|---|---|---|---|
| AFF3 | Age >60 | ASPSCR1 | BCL2 | BCL6 | BCORL1 |
| BHLHE22 | BTK | CARD11 | CCND2 | CD58 | CHEK2 |
| CIT | CREB3L2 | DST | ETS1 | EYA2 | FANCF |
| FZD6 | GAS5 | HMGA1 | HOXA9 | IRF4 | KDM5C |
| KLK2 | LFNG | LMO2 | MACROD1 | MALAT1 | MEF2B/ |
| | | | | | MEF2BNB-MEF2B |
| MFNG | MLLT4 | MTCP1 | TET2 mutation | TP53 mutation | MYC |
| PIM1 | POLD1 | PPP3CA | RABEP1 | RAD51B | RBM6 |
| RECQL4 | RHBDF2 | RLTPR | RTEL1-TNFRSF6B | SMAD3 | SPTBN1 |
| SRRM3 | ST6GAL1 | SULF1 | SYP | TEAD2 | TFAP2A |
| TGFBR3 | U2AF2 | ZIC2 | | | |

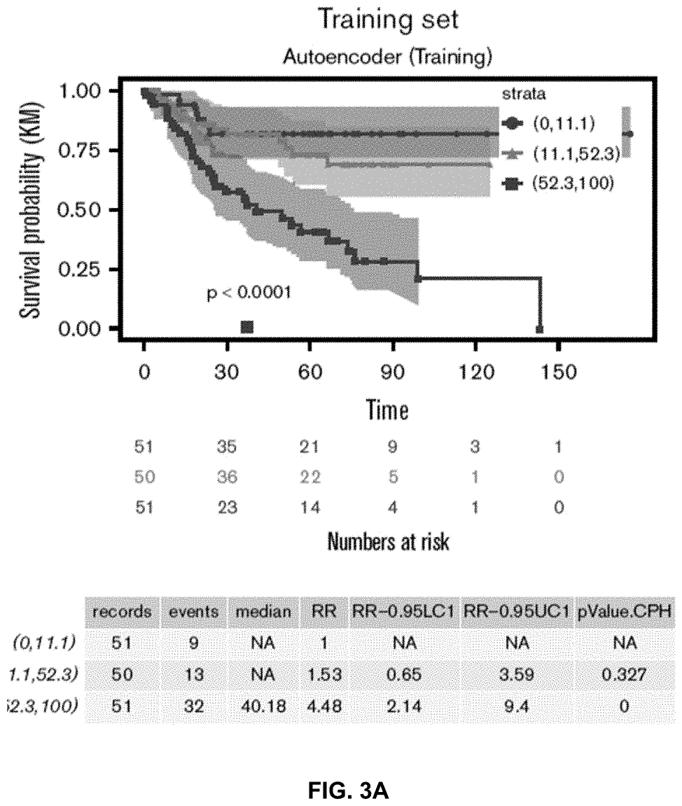

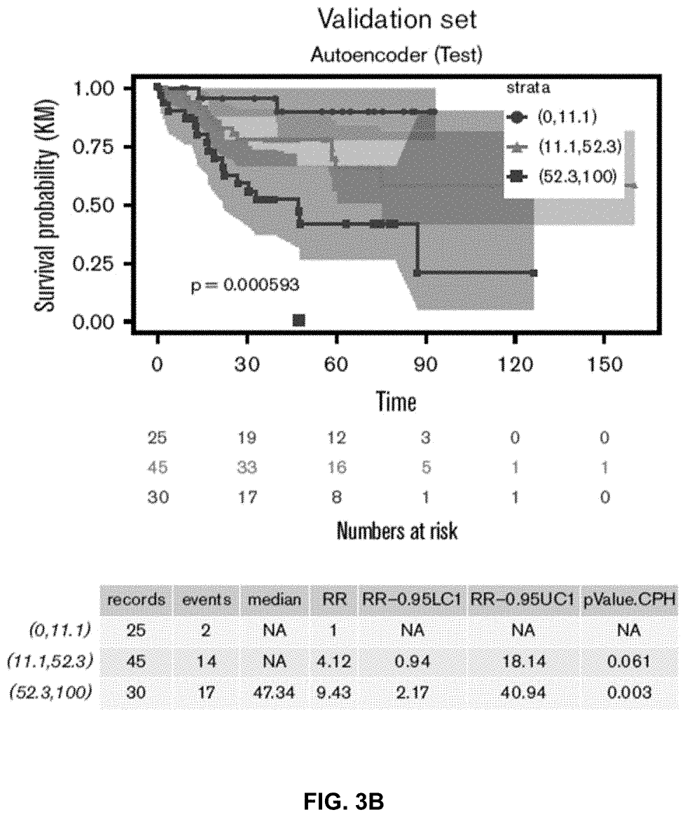

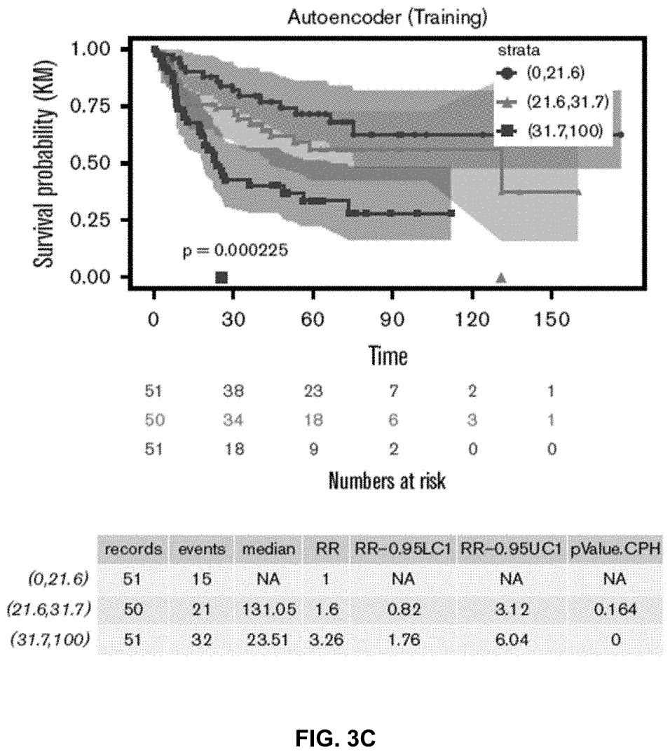

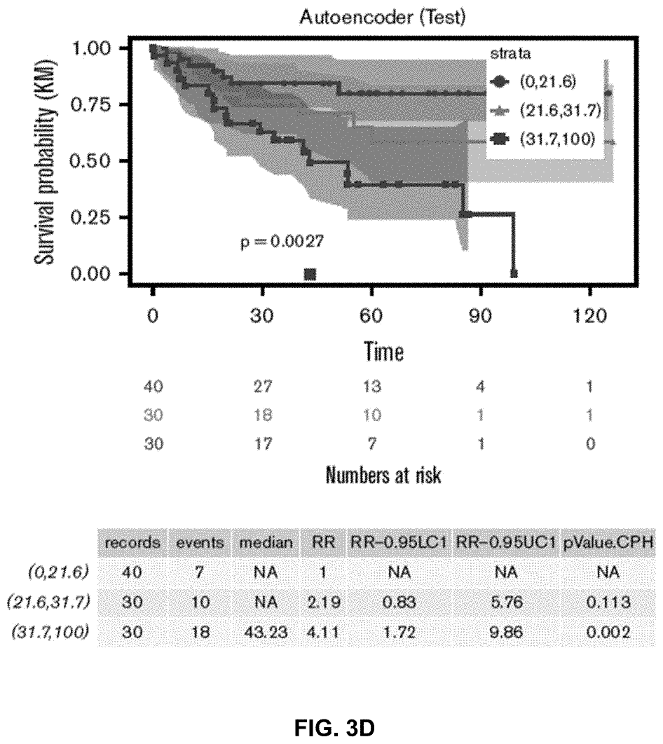

We used a similar neural network architecture as described for COO modeling and again included 2 neurons in the bottleneck layer to reduce the data into 2 dimensions (latent features). The top 7 variables contributing to the 2 latent features are age >60, TP53 mutation, CARD11 expression, BCL6 expression, MALAT1 expression, RABEP1 expression, and BCORL1 expression. A simple CPH model was built based on the 2 latent features obtained from the autoencoder (which are nonlinear combinations of the 57 variables) and provided a risk score (NGS-OS score) for each case, which was normalized to be between 0 (lowest risk) and 100 (highest risk). As shown in FIG. 3A-B, we divided the training set into 3 equal subgroups based on the NGS-OS risk score and found the high-risk group had strikingly poorer survival than the low- and intermediate-risk groups (P<0.0001). We then applied the NGS-OS model into the validation set and stratified patients using the same NGS-OS risk score cutoffs established in the training set and found that 3 resulting risk groups in the validation set showed incremental survival rates. The relative OS risk for the 3 subgroups was roughly 1, 4, and 9.

We followed a similar procedure to build a CPH model for PFS with 50 selected variables based on a 2-dimensional feature set obtained from an autoencoder (Table 5). The top 7 variables contributing to the model are TP53 mutation, CDK8 expression, LMO2 expression, BCR expression, TGFBR2 expression, CHD2 expression, and ETS1 expression. Although 24 variables are shared by the NGS-OS and NGS-PFS models, there are only 7 genes (AFF3, BCL6, CARD11, CCND2, IRF4, LMO2, and PIM1) shared by the NGS-COO and NGS-OS models and 5 genes (AFF3, BTLA, CREB3L2, FOXP1, and LMO2) shared by the NGS-COO and NGS-PFS models.

TABLE 5
List of 50 variables (49 for gene expression level and TP53 gene
mutation status) selected for building the NGS-PFS risk model

| | | | | | |
|---|---|---|---|---|---|
| AFF1 | AFF3 | ASPSCR1 | ATM | BCL2 | BCR |
| BTG2 | BTK | BTLA | CDK12 | CDK8 | CHD2 |
| CHEK2 | CIRH1A | CREB3L2 | DDIT3 | EDNRB | EPHB6 |
| ETS1 | FANCF | FOXP1 | FZD6 | GAB1 | GAS5 |
| GPR34 | IQCG | ITGA7 | KDM5C | KDSR | LAMA5 |
| LFNG | LIFR | LMO2 | MACROD1 | MAP2K5 | MFNG |
| TP53 mutation | MYC | NCSTN | NR6A1 | POU2AF1 | PRKCB |
| RLTPR | RPL22 | SHC2 | SMAD3 | SPTBN1 | ST6GAL1 |
| TEAD2 | TGFBR2 | | | | |

Similar with the NGS-OS risk scores, NGS-PFS risk scores identified one third of the training set and 30% of the validation set as high-risk patients (FIG. 3C-D). The relative risk for the low-, intermediate-, and high-risk groups in the validation sets was roughly 1, 2, and 4, respectively.

In this study, we developed novel DLBCL classification models based on both genetic and transcriptional variables derived from comprehensive RNA-Seq annotation and quantitative data. Our results demonstrated that both the NGS-COO classifier and NGS survival predictors were robust, and AI was able to assign COO/risk scores to new DLBCL cases (patients in the validation sets). Our NGS-COO classifier shared 8 genes with the 27-gene predictor by Wright et al (BCL6, CCND2, ETV6, IRF4, LMO2, LRMP, MYBL1, and PIM1),³¹ 8 genes with the 20-gene DLBCL Automatic classifier by Barrans et al (BCL6, CCND2, ETV6, FOXP1, IRF4, LMO2, LRMP, and PIM1),³⁰ 7 genes with the 14-gene-qNPA assay (BCL6, CCND2, IRF4, LMO2, LRMP, MYBL1 and PIM1),³⁵ and 3 genes with the NanoString Lymph2Cx assay (CREB3L2, MYBL1, and S1PR2).³⁸ Seven of the total 11 common genes (BCL6, CCND2, CREB3L2, FOXP1, IRF4, LMO2, and PIM1) are also shared by our NGS survival predictors, consistent with the association of COO with clinical outcome. The NGS-OS/PFS risk predictors had more significant P values in prognostic analysis than the NGS-COO classifier in the same patient cohort, suggesting that COO is only one of the biological contributors to DLBCL clinical outcome. However, the performance of NGS-OS/PFS risk predictors for other therapies is unknown. Different from previous COO/prognostic models, we integrated genetic abnormalities: MYD88 and EZH2 mutations in the NGS-COO classification model, TP53 and TET2 mutations in the NGS-OS risk model, and TP53 mutation in the NGS-PFS model.

The high-throughput RNA-Seq assays developed in this study using an NGS benchtop sequencer with approximately 3-day turnaround time have important practical implications. Although targeted NGS platforms have been implemented in the clinic to aid in diagnosis and therapeutic decisions,⁸⁰ and AI is emerging as an efficient tool in health care for large data processing and sophisticate model construction,^{76,81} currently no NGS panels and AI implementation have been developed for lymphoma diagnosis and management. Our study supports the reliability and practicality of using targeted NGS along with AI in generating clinically useful objective information. Compared with current IHC assays, DNA microarrays, and other GEP analysis techniques used for DLBCL COO classification, targeted RNA-Seq has a balanced advantage of genome-wide coverage, dynamic range of quantification, reproducibility, high throughput, and accuracy, as well as high sensitivity, automation, affordability, short assay time, and flexibility.⁸² As RNA-seq has become less costly and been integrated into clinical practice,⁸⁰ we expect that the generated RNA-seq data will be used not only to answer the COO and prognostic questions but also other diagnostic and clinical questions impacting clinical decisions, such as predicting clinical responses to novel therapies in clinic and in future prospective or retrospective studies.^{80,83}

The current proof-of-principle study demonstrates the potential utility of the targeted RNA-Seq assay for accurate and reproducible DLBCL-COO subclassification in daily clinical practice using a commercially available NGS platform; streamline analysis of high-throughput RNA-Seq data, COO assignment, and risk prediction by AI can further improve the workflow.

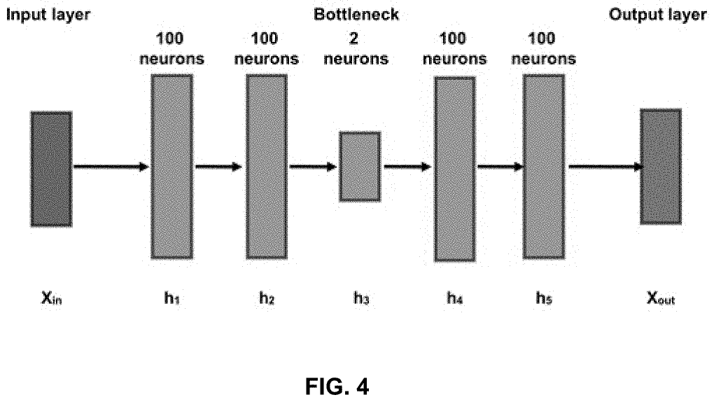

FIG. 4 schematically summarizes the deep learning approach used herein. In the example presented herein, the autoencoder took the 48 selected variables as input and passed them through hidden layers such that the output was a close approximation of the input data.

Here, the input data are denoted as Xᵢₙ, the hidden layers as h, and the output layer as Xₒᵤₜ. The model can be presented as follows:

h₀=xᵢₙ

hₗ=ƒₗ(hₗ₋₁Wₗ+bₗ),l=1, . . . ,5

h₆=xₒᵤₜ=g(h₅Wₒᵤₜ+bₒᵤₜ)

Where Wₗ is the weight matrix connecting layers l−1 and l, bₗ is the corresponding vector of biases, fₗ is a nonlinear transformation function, g is a link function that maps the last year to Xₒᵤₜ with the corresponding weight matrix Wₒᵤₜ and bias vector bₒᵤₜ.

The inventor set f to the hyperbolic tangent (tanh) function for all the hidden layers. Assuming that the output has Gaussian distribution, the inventor set the link function g to the identity function. The model parameters, W and b, are estimated such that Xₒᵤₜ becomes a close approximation (in terms of mean square error) of Xᵢₙ. More specifically, the H2O package in R was used to fit the model, and the corresponding code is presented below. To improve the generalizability of the model, the drop-out ratio from the input layer was set to 50% and the L2 regularization term was set to 0.01. The top 5 contributing variables to the model were Mute.MYD88, Mute.EZH2, RASGRF1, MYBL1, S1PR2, SSBP2, IRF4.
h2o.deeplearning(model_id=‘class.ae’, x=x.indep,
   training_frame=train,
   activation=“Tanh”,
   autoencoder=TRUE,
   hidden=c(100, 100, 2, 100, 100),
   input_dropout_ratio=0.5,
   l2=0.01,
   variable_importances=TRUE,
   export_weights_and_biases=TRUE,
   epochs=50)

### REFERENCES

1. Alizadeh A A, Eisen M B, Davis R E, et al. Distinct types of diffuse large B-cell lymphoma identified by gene expression profiling. Nature. 2000; 403(6769):503-511.
2. Lenz G, Wright G, Dave S S, et al; Lymphoma/Leukemia Molecular Profiling Project. Stromal gene signatures in large-B-cell lymphomas. N Engl J Med. 2008; 359(22):2313-2323.
3. Rosenwald A, Wright G, Chan W C, et al; Lymphoma/Leukemia Molecular Profiling Project. The use of molecular profiling to predict survival after chemotherapy for diffuse large-B-cell lymphoma. N Engl J Med. 2002; 346(25):1937-1947.
4. Vitolo U, Trněný M, Belada D, et al. Obinutuzumab or rituximab plus cyclophosphamide, doxorubicin, vincristine, and prednisone in previously untreated diffuse large B-cell lymphoma. J Clin Oncol. 2017; 35(31):3529-3537.
5. Thieblemont C, Briere J, Mounier N, et al. The germinal center/activated B-cell subclassification has a prognostic impact for response to salvage therapy in relapsed/refractory diffuse large B-cell lymphoma: a bio-CORAL study. J Clin Oncol. 2011; 29(31):4079-4087.
6. Castellino A, Chiappella A, LaPlant B R, et al. Lenalidomide plus R-CHOP21 in newly diagnosed diffuse large B-cell lymphoma (DLBCL): long-term follow-up results from a combined analysis from two phase 2 trials. Blood Cancer J. 2018; 8(11):108.
7. Czuczman M S, Trněný M, Davies A, et al. A phase 2/3 multicenter, randomized, open-label study to compare the efficacy and safety of lenalidomide versus investigator's choice in patients with relapsed or refractory diffuse large B-cell lymphoma. Clin Cancer Res. 2017; 23(15):4127-4137.
8. Goy A, Ramchandren R, Ghosh N, et al. Ibrutinib plus lenalidomide and rituximab has promising activity in relapsed/refractory non-germinal center B-cell-like DLBCL. Blood. 2019; 134(13):1024-1036.
9. Wilson W H, Young R M, Schmitz R, et al. Targeting B cell receptor signaling with ibrutinib in diffuse large B cell lymphoma. Nat Med. 2015; 21(8):922-926.
10. Ruan J, Martin P, Furman R R, et al. Bortezomib plus CHOP-rituximab for previously untreated diffuse large B-cell lymphoma and mantle cell lymphoma. J Clin Oncol. 2011; 29(6):690-697.
11. Herrera A F, Goy A, Mehta A, et al. Safety and activity of ibrutinib in combination with durvalumab in patients with relapsed or refractory follicular lymphoma or diffuse large B-cell lymphoma. Am J Hematol. 2020; 95(1):18-27.
12. Schneider C, Pasqualucci L, Dalla-Favera R. Molecular pathogenesis of diffuse large B-cell lymphoma. Semin Diagn Pathol. 2011; 28(2):167-177.
13. Davis R E, Ngo V N, Lenz G, et al. Chronic active B-cell-receptor signalling in diffuse large B-cell lymphoma. Nature. 2010; 463(7277):88-92.
14. Hu S, Xu-Monette Z Y, Tzankov A, et al. MYC/BCL2 protein coexpression contributes to the inferior survival of activated B-cell subtype of diffuse large B-cell lymphoma and demonstrates high-risk gene expression signatures: a report from The International DLBCL Rituximab-CHOP Consortium Program. Blood. 2013; 121(20):4021-4031, quiz 4250.
15. Mai Y, Yu J J, Bartholdy B, et al. An oxidative stress-based mechanism of doxorubicin cytotoxicity suggests new therapeutic strategies in ABC-DLBCL. Blood. 2016; 128(24):2797-2807.
16. Swerdlow S H, Campo E, Pileri S A, et al. The 2016 revision of the World Health Organization classification of lymphoid neoplasms. Blood. 2016; 127(20):2375-2390.
17. Colomo L, López-Guillermo A, Perales M, et al. Clinical impact of the differentiation profile assessed by immunophenotyping in patients with diffuse large B-cell lymphoma. Blood. 2003; 101(1):78-84.
18. Hans C P, Weisenburger D D, Greiner T C, et al. Confirmation of the molecular classification of diffuse large B-cell lymphoma by immunohistochemistry using a tissue microarray. Blood. 2004; 103(1):275-282.
19. de Jong D, Rosenwald A, Chhanabhai M, et al; Lunenburg Lymphoma Biomarker Consortium. Immunohistochemical prognostic markers in diffuse large B-cell lymphoma: validation of tissue microarray as a prerequisite for broad clinical applications—a study from the Lunenburg Lymphoma Biomarker Consortium. J Clin Oncol. 2007; 25(7):805-812.
20. Muris J J, Meijer C J, Vos W, et al. Immunohistochemical profiling based on Bcl-2, CD10 and MUM1 expression improves risk stratification in patients with primary nodal diffuse large B cell lymphoma. J Pathol. 2006; 208(5):714-723.
21. Choi W W, Weisenburger D D, Greiner T C, et al. A new immunostain algorithm classifies diffuse large B-cell lymphoma into molecular subtypes with high accuracy. Clin Cancer Res. 2009; 15(17):5494-5502.
22. Meyer P N, Fu K, Greiner T C, et al. Immunohistochemical methods for predicting cell of origin and survival in patients with diffuse large B-cell lymphoma treated with rituximab. J Clin Oncol. 2011; 29(2):200-207.
23. Gutiérrez-García G, Cardesa-Salzmann T, Climent F, et al; Grup per l'Estudi dels Limfomes de Catalunya I Balears (GELCAB). Gene-expression profiling and not immunophenotypic algorithms predicts prognosis in patients with diffuse large B-cell lymphoma treated with immunochemotherapy. Blood. 2011; 117(18):4836-4843.
24. Visco C, Li Y, Xu-Monette Z Y, et al. Comprehensive gene expression profiling and immunohistochemical studies support application of immunophenotypic algorithm for molecular subtype classification in diffuse large B-cell lymphoma: a report from the International DLBCL Rituximab-CHOP Consortium Program Study [published correction appears in Leukemia 2014; 28:980]. Leukemia. 2012; 26(9):2103-2113.
25. Molina T J, Canioni D, Copie-Bergman C, et al. Young patients with non-germinal center B-cell-like diffuse large B-cell lymphoma benefit from intensified chemotherapy with ACVBP plus rituximab compared with CHOP plus rituximab: analysis of data from the Groupe d'Etudes des Lymphomes de l'Adulte/lymphoma study association phase III trial LNH 03-2B. J Clin Oncol. 2014; 32(35):3996-4003.
26. Ott G, Ziepert M, Klapper W, et al. Immunoblastic morphology but not the immunohistochemical GCB/nonGCB classifier predicts outcome in diffuse large B-cell lymphoma in the RICOVER-60 trial of the DSHNHL. Blood. 2010; 116(23):4916-4925.
27. Moskowitz C H, Zelenetz A D, Kewalramani T, et al. Cell of origin, germinal center versus nongerminal center, determined by immunohistochemistry on tissue microarray, does not correlate with outcome in patients with relapsed and refractory DLBCL. Blood. 2005; 106(10): 3383-3385.
28. Saad A G, Grada Z, Bishop B, et al. nCounter NanoString assay shows variable concordance with immunohistochemistry-based algorithms in classifying cases of diffuse large B-cell lymphoma according to the cell-of-origin. Appl Immunohistochem Mol Morphol. 2019; 27(9):644-648.
29. Williams P M, Li R, Johnson N A, Wright G, Heath J D, Gascoyne R D. A novel method of amplification of FFPET-derived RNA enables accurate disease classification with microarrays. J Mol Diagn. 2010; 12(5):680-686.
30. Barrans S L, Crouch S, Care M A, et al. Whole genome expression profiling based on paraffin embedded tissue can be used to classify diffuse large B-cell lymphoma and predict clinical outcome. Br J Haematol. 2012; 159(4):441-453.
31. Wright G, Tan B, Rosenwald A, Hurt E H, Wiestner A, Staudt L M. A gene expression-based method to diagnose clinically distinct subgroups of diffuse large B cell lymphoma. Proc Natl Acad Sci USA. 2003; 100(17):9991-9996.
32. Martel R R, Botros 1W, Rounseville M P, et al. Multiplexed screening assay for mRNA combining nuclease protection with luminescent array detection. Assay Drug Dev Technol. 2002; 1(1 Pt 1):61-71.
33. Qi Z, Wang L, He A, Ma-Edmonds M, Cogswell J. Evaluation and selection of a non-PCR based technology for improved gene expression profiling from clinical formalin-fixed, paraffin-embedded samples. Bioanalysis. 2016; 8(22):2305-2316.
34. Roberts R A, Sabalos C M, LeBlanc M L, et al. Quantitative nuclease protection assay in paraffin-embedded tissue replicates prognostic microarray gene expression in diffuse large-B-cell lymphoma. Lab Invest. 2007; 87(10):979-997.
35. Rimsza L M, Wright G, Schwartz M, et al. Accurate classification of diffuse large B-cell lymphoma into germinal center and activated B-cell subtypes using a nuclease protection assay on formalin-fixed, paraffin-embedded tissues. Clin Cancer Res. 2011; 17(11):3727-3732.
36. Younes A, Sehn L H, Johnson P, et al; PHOENIX investigators. Randomized phase III trial of ibrutinib and rituximab plus cyclophosphamide, doxorubicin, vincristine, and prednisone in non-germinal center B-cell diffuse large B-cell lymphoma. J Clin Oncol. 2019; 37(15):1285-1295.
37. Geiss G K, Bumgarner R E, Birditt B, et al. Direct multiplexed measurement of gene expression with color-coded probe pairs [published correction appears in Nat Biotechnol 2008; 26:709]. Nat Biotechnol. 2008; 26(3):317-325.
38. Scott D W, Wright G W, Williams P M, et al. Determining cell-of-origin subtypes of diffuse large B-cell lymphoma using gene expression in formalin-fixed paraffin-embedded tissue. Blood. 2014; 123(8):1214-1217.
39. Masqué-Soler N, Szczepanowski M, Kohler C W, Spang R, Klapper W. Molecular classification of mature aggressive B-cell lymphoma using digital multiplexed gene expression on formalin-fixed paraffin-embedded biopsy specimens. Blood. 2013; 122(11):1985-1986.
40. Szczepanowski M, Lange J, Kohler C W, et al. Cell-of-origin classification by gene expression and MYC-rearrangements in diffuse large B-cell lymphoma of children and adolescents. Br J Haematol. 2017; 179(1):116-119.
41. Cascione L, Rinaldi A, Chiappella A, et al. Diffuse large B cell lymphoma cell of origin by digital expression profiling in the REAL07 Phase 1-2 study. Br J Haematol. 2018; 182(3):453-456.
42. Klanova M, Sehn L H, Bence-Bruckler I, et al. Integration of cell of origin into the clinical CNS International Prognostic Index improves CNS relapse prediction in DLBCL. Blood. 2019; 133(9):919-926.
43. Bolen C R, Klanova M, Trneny M, et al. Prognostic impact of somatic mutations in diffuse large B-cell lymphoma and relationship to cell-of-origin: data from the phase III GOYA study. Haematologica. 2019; haematol. 2019.227892.
44. Nowakowski G S, Chiappella A, Witzig T E, et al. Variable global distribution of cell of-origin from the ROBUST phase 3 study in diffuse large B-cell lymphoma. Haematologica. 2020; 105(2):e72-e75.
45. King R L, Nowakowski G S, Witzig T E, et al. Rapid, real time pathology review for ECOG/ACRIN 1412: a novel and successful paradigm for future lymphoma clinical trials in the precision medicine era. Blood Cancer J. 2018; 8(3):27.
46. Veldman-Jones M H, Lai Z, Wappett M, et al. Reproducible, quantitative, and flexible molecular subtyping of clinical DLBCL samples using the NanoString nCounter system. Clin Cancer Res. 2015; 21(10):2367-2378.
47. Scott D W, Mottok A, Ennishi D, et al. Prognostic significance of diffuse large B-cell lymphoma cell of origin determined by digital gene expression in formalin-fixed paraffin-embedded tissue biopsies. J Clin Oncol. 2015; 33(26):2848-2856.
48. Abdulla M, Hollander P, Pandzic T, et al. Cell-of-origin determined by both gene expression profiling and immunohistochemistry is the strongest predictor of survival in patients with diffuse large B-cell lymphoma. Am J Hematol. 2020; 95(1):57-67.
49. Kendrick S, Tus K, Wright G, et al. Diffuse large B-cell lymphoma cell-of-origin classification using the Lymph2Cx assay in the context of BCL2 and MYC expression status. Leuk Lymphoma. 2016; 57(3):717-720.
50. Phang K C, Akhter A, Tizen N M S, et al. Comparison of protein-based cell-of-origin classification to the Lymph2Cx RNA assay in a cohort of diffuse large B-cell lymphomas in Malaysia. J Clin Pathol. 2018; 71(3):215-220.
51. Jais J P, Molina T J, Ruminy P, et al. Reliable subtype classification of diffuse large B-cell lymphoma samples from GELA LNH2003 trials using the Lymph2Cx gene expression assay. Haematologica. 2017; 102(10):e404-e406.
52. Staiger A M, Ziepert M, Horn H, et al; German High-Grade Lymphoma Study Group. Clinical impact of the cell-of-origin classification and the MYC/BCL2 dual expresser status in diffuse large b-cell lymphoma treated within prospective clinical trials of the German High-Grade Non-Hodgkin's Lymphoma Study Group. J Clin Oncol. 2017; 35(22):2515-2526.
53. Hwang H S, Yoon D H, Hong J Y, et al. The cell-of-origin classification of diffuse large B cell lymphoma in a Korean population by the Lymph2Cx assay and its correlation with immunohistochemical algorithms. Ann Hematol. 2018; 97(12):2363-2372.
54. Schouten J P, McElgunn C J, Waaijer R, Zwijnenburg D, Diepvens F, Pals G. Relative quantification of 40 nucleic acid sequences by multiplex ligation-dependent probe amplification. Nucleic Acids Res. 2002; 30(12):e57.
55. Mareschal S, Ruminy P, Bagacean C, et al. Accurate classification of germinal center B-cell-like/activated B-cell-like diffuse large B-cell lymphoma using a simple and rapid reverse transcriptase-multiplex ligation-dependent probe amplification assay: a CALYM study. J Mol Diagn. 2015; S1525-1578(15) 00046-X.
56. Bob'ee V, Ruminy P, Marchand V, et al. Determination of molecular subtypes of diffuse large B-cell lymphoma using a reverse transcriptase multiplex ligation-dependent probe amplification classifier: a CALYM study. J Mol Diagn. 2017; 19(6):892-904.
57. Lossos I S, Czerwinski D K, Alizadeh A A, et al. Prediction of survival in diffuse large-B-cell lymphoma based on the expression of six genes. N Engl J Med. 2004; 350(18):1828-1837.
58. Shipp M A, Ross K N, Tamayo P, et al. Diffuse large B-cell lymphoma outcome prediction by gene-expression profiling and supervised machine learning. Nat Med. 2002; 8(1):68-74.
59. Leonard J P, Kolibaba K S, Reeves J A, et al. Randomized phase II study of R-CHOP with or without bortezomib in previously untreated patients with non-germinal center B-cell-like diffuse large B-cell lymphoma. J Clin Oncol. 2017; 35(31):3538-3546.
60. Davies A, Cummin T E, Barrans S, et al. Gene-expression profiling of bortezomib added to standard chemoimmunotherapy for diffuse large B-cell lymphoma (REMoDL-B): an open-label, randomised, phase 3 trial. Lancet Oncol. 2019; 20(5):649-662.
61. Schmitz R, Wright G W, Huang D W, et al. Genetics and pathogenesis of diffuse large B-cell lymphoma. N Engl J Med. 2018; 378(15):1396-1407.
62. Chapuy B, Stewart C, Dunford A J, et al. Molecular subtypes of diffuse large B cell lymphoma are associated with distinct pathogenic mechanisms and outcomes [published correction appears in Nat Med 2018; 24:1290-1292]. Nat Med. 2018; 24(5):679-690.
63. Amin A D, Peters T L, Li L, et al. Diffuse large B-cell lymphoma: can genomics improve treatment options for a curable cancer? Cold Spring Harb Mol Case Stud. 2017; 3(3):a001719.
64. Reddy A, Zhang J, Davis N S, et al. Genetic and functional drivers of diffuse large B cell lymphoma. Cell. 2017; 171:481-494.
65. Dubois S, Tesson B, Mareschal S, et al; Lymphoma Study Association (LYSA) investigators. Refining diffuse large B-cell lymphoma subgroups using integrated analysis of molecular profiles. EBioMedicine. 2019; 48:58-69.
66. Wright G W, Wilson W H, Staudt L M. Genetics of diffuse large B-cell lymphoma. N Engl J Med. 2018; 379(5):493-494.
67. Arthur S E, Jiang A, Grande B M, et al. Genome-wide discovery of somatic regulatory variants in diffuse large B-cell lymphoma. Nat Commun. 2018; 9(1): 4001.
68. Wright G W, Huang D W, Phelan J D, et al. A probabilistic classification tool for genetic subtypes of diffuse large B cell lymphoma with therapeutic implications. Cancer Cell. 2020; 37:551-568.
69. Xu-Monette Z Y, Wu L, Visco C, et al. Mutational profile and prognostic significance of TP53 in diffuse large B-cell lymphoma patients treated with R-CHOP: report from an International DLBCL Rituximab-CHOP Consortium Program Study. Blood. 2012; 120(19):3986-3996.
70. Xu-Monette Z Y, Li L, Byrd J C, et al. Assessment of CD37 B-cell antigen and cell of origin significantly improves risk prediction in diffuse large B-cell lymphoma. Blood. 2016; 128(26):3083-3100.
71. Xu-Monette Z Y, Xiao M, Au Q, et al. Immune profiling and quantitative analysis decipher the clinical role of immune-checkpoint expression in the tumor immune microenvironment of DLBCL. Cancer Immunol Res. 2019; 7(4):644-657.
72. Irizarry R A, Bolstad B M, Collin F, Cope L M, Hobbs B, Speed T P. Summaries of Affymetrix GeneChip probe level data. Nucleic Acids Res. 2003; 31(4): e15.
73. Tusher V G, Tibshirani R, Chu G. Significance analysis of microarrays applied to the ionizing radiation response. Proc Natl Acad Sci USA. 2001; 98(9):5116-5121.
74. Eisen M B, Spellman P T, Brown P O, Botstein D. Cluster analysis and display of genome-wide expression patterns. Proc Natl Acad Sci USA. 1998; 95(25):14863-14868.
75. Eraslan G, Avsec Ž, Gagneur J, Theis F J. Deep learning: new computational modelling techniques for genomics. Nat Rev Genet. 2019; 20(7):389-403.
76. Ching T, Himmelstein D S, Beaulieu-Jones B K, et al. Opportunities and obstacles for deep learning in biology and medicine. J R Soc Interface. 2018; 15(141):15.
77. Sha C, Barrans S, Cucco F, et al. Molecular high-grade B-cell lymphoma: defining a poor-risk group that requires different approaches to therapy. J Clin Oncol. 2019; 37(3):202-212.
78. Ennishi D, Jiang A, Boyle M, et al. Double-hit gene expression signature defines a distinct subgroup of germinal center B-cell-like diffuse large B-cell lymphoma. J Clin Oncol. 2019; 37(3):190-201.
79. Bojarczuk K, Wienand K, Ryan J A, et al. Targeted inhibition of PI3Ka/d is synergistic with BCL-2 blockade in genetically defined subtypes of DLBCL. Blood. 2019; 133(1):70-80.
80. Oberg J A, Glade Bender J L, Sulis M L, et al. Implementation of next generation sequencing into pediatric hematology-oncology practice: moving beyond actionable alterations. Genome Med. 2016; 8(1):133.
81. Du-Harpur X, Watt F M, Luscombe N M, Lynch M D. What is AI? Applications of artificial intelligence to dermatology. Br J Dermatol. 2020; bjd.18880.
82. Narrandes S, Xu W. Gene expression detection assay for cancer clinical use. J Cancer. 2018; 9(13):2249-2265.
83. Zhang W, Yu Y, Hertwig F, et al. Comparison of RNA-seq and microarray-based models for clinical endpoint prediction. Genome Biol. 2015; 16(1):133.
84. Xu-Monette Z Y, et al. A refined cell-of-origin classifier with targeted NGS and artificial intelligence shows robust predictive value in DLBCL. Blood Advances 2020; 14(4):3391.

What is claimed is:

1. A method of treating diffuse large B-cell lymphoma, comprising:
obtaining a sample from a patient having diffuse large B-cell lymphoma;
detecting in the sample, by an assay, mutation in each gene in a first panel;
quantifying in the sample an expression level of each gene in a second panel;
classifying the diffuse large B-cell lymphoma of the patient as having a cell of origin of either (i) germinal-center B-cell-like or (ii) activated B-cell-like; and
treating the patient with a cancer treatment therapy regime;
wherein the first panel comprises at least one gene selected from the group consisting of EZH1 and MYD88; and
the second panel comprises at least one gene selected from the group consisting of IRF4, MYBL1, RASGRF1, S1PR2 and SSBP2.

2. The method of claim 1, wherein the first panel comprises EZH1 and MYD88, and the second panel comprises IRF4, MYBL1, RASGRF1, S1PR2 and SSBP2.

3. The method of claim 1, wherein first panel comprises EZH1 and MYD88, and the second panel comprises AFF3, AHR, AUTS2, BCAS4, BCL6, BTLA, CARD11, CCND2, CCND3, CD22, CD44, COL9A3, CREB3L2, EBF1, ETV6, FAM46C, FOXP1, IKZF1, IL2RA, IRF4, IRS1, KANK1, LCK, LMO2, LPP, LRMP, LRP5, LRRK2, LYL1, LYN, METTL7B, MYBL1, P2RY8, PAG1, PAK6, PDGFD, PIK3CG, PIM1, PTK2, PTK2B, PTPN2, RASGRF1, S1PR2, SSBP2, STAT3 and TBL1XR1.

4. The method of claim 1, wherein the detecting in the sample mutation in each gene in the first panel, and quantifying in the sample the expression level of each gene in the second panel, is carried out by performing next generation sequencing in a single assay.

5. The method of claim 1, wherein the treating of the patient with the cancer treatment therapy regime comprises administering at least one therapy selected from the group consisting of cyclophosphamide, doxorubicin, vincristine, prednisone, rituximab, obinutuzumab, dexamethasone, cytarabine, cisplatin, lenalidomide, ibrutinib, bortezomib, durvalumab and autologous stem cell transplantation.

6. The method of claim 1, wherein the confidence of classifying the diffuse large B-cell lymphoma of the patient as having the cell of origin of either (i) germinal-center B-cell-like or (ii) activated B-cell-like is a probability of at least 0.8.

7. A method, comprising:
detecting in a sample from a patient having diffuse large B-cell lymphoma, by an assay, mutation in each gene in a first panel; and
quantifying in the sample an expression level of each gene in a second panel;
wherein the first panel comprises at least one gene selected from the group consisting of EZH1 and MYD88; and
the second panel comprises at least one gene selected from the group consisting of IRF4, MYBL1, RASGRF1, S1PR2 and SSBP2.

8. The method of claim 7, wherein the first panel comprises EZH1 and MYD88, and the second panel comprises IRF4, MYBL1, RASGRF1, S1PR2 and SSBP2.

9. The method of claim 7, wherein first panel comprises EZH1 and MYD88, and the second panel comprises AFF3, AHR, AUTS2, BCAS4, BCL6, BTLA, CARD11, CCND2, CCND3, CD22, CD44, COL9A3, CREB3L2, EBF1, ETV6, FAM46C, FOXP1, IKZF1, IL2RA, IRF4, IRS1, KANK1, LCK, LMO2, LPP, LRMP, LRP5, LRRK2, LYL1, LYN, METTL7B, MYBL1, P2RY8, PAG1, PAK6, PDGFD, PIK3CG, PIM1, PTK2, PTK2B, PTPN2, RASGRF1, S1PR2, SSBP2, STAT3 and TBL1XR1.

10. The method of claim 7, wherein the detecting in the sample mutation in each gene in the first panel, and quantifying in the sample the expression level of each gene in the second panel, is carried out by performing next generation sequencing in a single assay.

11. The method of claim 7, further comprising treating the patient with a cancer treatment therapy regime comprises administering at least one therapy selected from the group consisting of cyclophosphamide, doxorubicin, vincristine, prednisone, rituximab, obinutuzumab, dexamethasone, cytarabine, cisplatin, lenalidomide, ibrutinib, bortezomib, durvalumab and autologous stem cell transplantation.

12. The method of claim 7, wherein the sample is formaldehyde-fixed paraffin-embedded tissue.

13. A method, comprising:
detecting in a sample from a patient having diffuse large B-cell lymphoma, by an assay, mutation in each gene in a first panel; and
quantifying in the sample an expression level of each gene in a second panel;
wherein the first panel comprises TP53; and
the second panel comprises at least one gene selected from the group consisting of CARD11, BCL6, MALAT1, RABEP1 and BCORL1.

14. The method of claim 13, wherein the second panel comprises CARD11, BCL6, MALAT1, RABEP1 and BCORL1.

15. The method of claim 13, wherein the first panel comprises TP53 and TET2, and the second panel comprises AFF3, ASPSCR1, BCL2, BCL6, BCORL1, BHLHE22, BTK, CARD11, CCND2, CD58, CHEK2, CIT, CREB3L2, DST, ETS1, EYA2, FANCF, FZD6, GAS5, HMGA1, HOXA9, IRF4, KDM5C, KLK2, LFNG, LMO2, MACROD1, MALAT1, MEF2B/MEF2BNB-MEF2B, MFNG, MLLT4, MTCP1, MYC, PIM1, POLD1, PPP3CA, RABEP1, RAD51B, RBM6, RECQL4, RHBDF2, RLTPR, RTEL1-TNFRSF6B, SMAD3, SPTBN1, SRRM3, ST6GAL1, SULF1, SYP, TEAD2, TFAP2A, TGFBR3, U2AF2 and ZIC2.

16. The method of claim 13, wherein the detecting in the sample mutation in each gene in the first panel, and quantifying in the sample the expression level of each gene in the second panel, is carried out by performing next generation sequencing in a single assay.

17. The method of claim 13, wherein the sample is formaldehyde-fixed paraffin-embedded tissue.

18. A method, comprising:
detecting in a sample from a patient having diffuse large B-cell lymphoma, by an assay, mutation in each gene in a first panel; and
quantifying in the sample an expression level of each gene in a second panel;
wherein the first panel comprises TP53; and
the second panel comprises at least one gene selected from the group consisting of CDK8, LMO2, BCR, TGFBR2, CHD2 and ETS1.

19. The method of claim 18, wherein the second panel comprises CDK8, LMO2, BCR, TGFBR2, CHD2 and ETS1.

20. The method of claim 18, wherein the second panel comprises AFF1, AFF3, ASPSCR1, ATM, BCL2, BCR, BTG2, BTK, BTLA, CDK12, CDK8, CHD2, CHEK2, CIRH1A, CREB3L2, DDIT3, EDNRB, EPHB6, ETS1, FANCF, FOXP1, FZD6, GAB1, GAS5, GPR34, IQCG, ITGA7, KDM5C, KDSR, LAMA5, LFNG, LIFR, LMO2, MACROD1, MAP2K5, MFNG, MYC, NCSTN, NR6A1, POU2AF1, PRKCB, RLTPR, RPL22, SHC2, SMAD3, SPTBN1, ST6GAL1, TEAD2 and TGFBR2.

21. The method of claim 18, wherein the detecting in the sample mutation in each gene in the first panel, and quantifying in the sample the expression level of each gene in the second panel, is carried out by performing next generation sequencing in a single assay.

22. The method of claim 18, wherein the sample is formaldehyde-fixed paraffin-embedded tissue.
